(19) United States
(12) Patent Application Publication
(10) Pub. No.: US 2005/0065534 A1
(43) Pub. Date: Mar. 24, 2005

(54) Set for implanting an intra-ocular lens

(76) Inventor: Emil Hohl, Heerburg (CH)

Correspondence Address: NIXON & VANDERHYE, PC, 1100 N GLEBE ROAD, 8TH FLOOR, ARLINGTON, VA, 22201-4714, US

(21) Appl. No.: 10/497,184

(22) PCT Filed: Nov. 29, 2002
(86) PCT No.: PCT/CH02/00646

(30) Foreign Application Priority Data
Nov. 30, 2001 (CH) 2195/01
May 8, 2002 (CH) 792/02
May 8, 2002 (CH) 793/02
Aug. 28, 2002 (CH) 1468/02

(51) Int. Cl. A61F 9/013

(52) U.S. Cl. 606/107

(57) ABSTRACT

In order to introduce an intra-ocular lens into the capsular sac of the eye, excess pressure is generated to propel a lens that is floating in a lubricant out of the injector nozzle. The compressible, deformable plunger can adapt to the nozzle channel that tapers towards the front. The lens becomes more folded as it continues along its path, having an extremely small diameter when it reaches the end of said path. The incision can thus be extremely small. The kit comprises a lens carrier and a lens. The lens is held in the lens carrier in a stress-free manner. The lens and lens carrier are preferably held by a support and are packed in sterile packaging until required for use, in the case of a hydrophilic lens in a liquid that protects said lens from drying out. During the operation, the lens carrier, together with the lens stored therein, is removed from the packaging, folded and inserted into the injector. The channel is then filled with a viscoelastic liquid. The lens can then be injected into the capsular sac of the eye to be treated. There is thus no risk of the lens being damaged during the folding process and the injection.

The present invention relates to a method for inserting an intraocular lens (IOL) and a set for containing and/or inserting an intraocular lens.

In cataract operations it has nowadays become a standard practice to insert artificial lenses, so-called intraocular lenses, into the capsular bag of the eye. The operation consists of making an ocular incision, typically of the order of 3 to 4 mm, through which the natural crystalline lens is first removed and the implant is then inserted. The removal of the natural crystalline lens is effected by first destroying it by means of ultrasound and then sucking it away. Once the natural crystalline lens has been removed, the lens is inserted through the incision into the capsular bag in a folded condition. As soon as the folded lens has been introduced into the capsular bag, it unfolds back into its original form.

Improved operating instruments and implants enable the surgeon to make the incisions clearly smaller. Today the removal of the natural crystalline lens can already be effected through incisions of the order of 2 mm. But this makes sense only if the intraocular lens can likewise be inserted through the same incision.

Recent years have seen the development of lens carriers or cartridges for the insertion of an intraocular lens into which a lens can be loaded and then ejected from the lens carrier by means an injector. Examples of such lens carriers are known from the American patents U.S. Pat. No. 6,267,768, U.S. Pat. No. 5,810,833, U.S. Pat. No. 6,283,975 and U.S. Pat. No. 6,248,111. The aforesaid citations are hereby received by reference into the present application.

The injector is an operating instrument with a casing and an axially movable plunger contained in the casing. A cut-out, a slot for example, is provided in the jacket of the casing into which a lens carrier can be loaded. Most of the time the lens carrier has a cylindrical loading channel terminating with an injector nozzle that tapers in the direction of the tip (distal end of the lens carrier). The lens carrier is held in the injector in such a manner that the plunger is aligned with the loading channel. When it is moved forward, the plunger penetrates into the loading channel and pushes the lens out of the injector nozzle.

Since there is a tendency towards ever smaller incisions, the necessary operating and implantation instruments have also undergone a corresponding miniaturization. Some injector tips nowadays have a diameter of less than 2 mm. For implantation purposes the lens has to be pressed through this small opening. It is self-evident that the lens has to be greatly compressed for this purpose and that this calls for a correspondingly great mechanical pressure.

In conventional injection systems the plunger of the injector comes into direct contact with the lens when the latter is implanted. Given the necessary strong compression of the lens, the more recent injection systems with injector nozzles having a diameter of less than 2 mm are clearly associated with the danger that the lens will be damaged during the implantation. Various solutions have therefore already been proposed with a view to avoiding damage to the lens during implantation.

U.S. Pat. No. 6,248,111, for example, suggests that the interior wall of the injector nozzle should be provided with a surface structure, for example grooves or similar, so that the lens will come into contact with only a part of the wall as it passes through the injection channel. In this way it is proposed to reduce the friction between the interior wall and the lens and, consequently, reduce the necessary ejection force. U.S. Pat. No. 6,283,975 suggests an injector system with improved lubricity properties. To this end a water-soluble lubricity agent, a surfactant for example, is physically bonded to the plasma-treated interior wall of the injector nozzle. The bonding of the lubricant is effected in manner different from covalent chemical bonding. The presence of the lubricant on the interior wall is intended to reduce the force needed for ejecting the lens.

With a view to avoiding damage to the lens during the implantation, it has also been suggested that the injector plunger, which is generally made of metal, should not be allowed to act directly on the lens and that a plastic buffer should be inserted between the plunger and the lens. Since the plastic buffer is softer than the plunger, there is a smaller risk of the lens being damaged when it comes into contact with it.

The intraocular lenses that are most widely used today consist of a hydrophilic material, generally on an acryl basis. Such lenses are supplied by their makers in a liquid bath within a sterile package. Conservation in a liquid is necessary in order to protect the lens against drying. During the operation the lens has to be removed from the package in a sterile region and inserted into the cartridge, after which this cartridge has to be loaded into the injector. Since these lenses constitute very small and springy structures, their insertion into the cartridge is associated with the danger that the lens will be dropped or slip away when it is folded and thus lose its sterility. Furthermore, these lenses are very sensitive structures that can easily be damaged when they are folded or transferred into a cartridge. The damage danger is particularly great as regards the so-called haptic that surrounds the optical part of the lens. It is also possible for the optical part to be damaged by the forceps when it is inserted into the cartridge. This danger is particularly great, because as a general rule the lens has not only to be inserted in the cartridge, but must also be folded while it is being inserted. The need for filling the lens carrier or cartridge during the operation and the handling errors associated therewith are responsible for the greater part of the complications during or after injection.

It is the object of the present invention to suggest a method and an improved lens carrier with which the disadvantages mentioned above can be substantially eliminated. In particular, the invention seeks to suggest a method with which the danger of damaging the lens during the implantation is clearly reduced. Furthermore, it is seeks to propose a set of instruments that makes it possible to implant a lens through an injector nozzle opening of less than 2 mm.

According to the invention, this object is attained by means of a method in which the cartridge, for the purposes of ejecting the intraocular lens from the lens carrier, produces an overpressure from the side of its proximal opening and the lens, together with at least a part of the lubricant, is pushed from the loading channel into the injector nozzle and eventually ejected therefrom. The method in accordance with the invention has the advantage that the lens can be ejected from the cartridge without any direct contact or, at least, by means of a greatly reduced contact with the plunger. In this connection the lubricant performs the additional function of a hydraulic means. The hydraulic means transfers the pressure exerted by a displacement element, a piston for example, to the lens. Consequently, the method is very protective and damage to the lens is avoided during the implantation. Another advantage is that with this method it is possible to press a lens without damage through openings as small as about 1.5 mm diameter, or even smaller. The overpressure may be produced by means of, for example, a pressure source, possibly a pump or a compressed-air cylinder, or mechanically by means of a displacement element, a piston for example. In the latter case an elastic and compressible material between the piston jacket and the interior wall of the loading channel can assure an at least liquid-tight seal, so that a displacement of the piston will cause the pressure to be transferred to the lens via the lubricant present.

In basic principle, known injectors may be used for carrying out the method. To this end the lens carrier with the lens may be loaded into an injector provided with a mobile plunger and a displacement element serving as piston may be inserted or introduced into the proximal opening of the loading channel. A movement of the displacement element, a piston for example, produces an overpressure that displaces the lens floating in the lubricant into the injector nozzle and eventually ejects it therefrom. The fact that known injectors can be used is yet another advantage of the method. Advantageously, the quantity of lubricant used is such that the lens will be enveloped by a plug of lubricant. The plug of lubricant assures a small ejection resistance, because it assures a uniform lubrication on all sides. Particularly advantageous is the fact that the novel injection procedure assures the simultaneous displacement of the lens and the lubricant.

It is advantageous to attach the displacement element to the plunger of an injector and then, after the loaded cartridge has been inserted in the injector, introduce it through the opening of the loading channel. Alternatively, the displacement element may already be inserted in the loading channel behind the lens when the lens carrier is loaded. Advantageously, the lens will be arranged at a certain distance from the displacement element and embedded in the lubricant, so that a buffer zone consisting of lubricant and possibly a little air is created between the lens and the displacement element.

The invention also relates to a set, especially a lens carrier set, capable of accommodating a lens with a lens carrier with a loading channel provided with an opening for a lens and an injector nozzle and a preferably axially aligned injector nozzle at the terminal of the loading channel, as well as means for the production of an overpressure that act through the opening. The overpressure means may such as to be capable of being attached to or inserted into the opening of the loading channel. The set in accordance with the invention has the advantage that a lens may be implanted in a well protected manner, especially without any direct or, at least, greatly reduced contact with an injection plunger. Such overpressure sources as pumps or compressed-air cylinders may be considered as overpressure means, as also a piston that can be inserted in the loading channel to provide a seal. Inserted in a conventional injector, the latter can be displaced by means of the mobile plunger present therein. When use is made of an elastic and especially a compressible material, preferably a plastic material, the displacement element may also be partly slid into the injector nozzle. Such materials as silicone or soft-elastic or foamed plastic materials have proved successful. Advantageously, the displacement element and/or the material of which it is made will have a hardness between 10 and 80 Shore, preferably between 20 and 65 Shore, and even more preferably between 25 and 40 Shore. The displacement element is therefore preferably soft-elastic and can adapt to the inner diameter of the loading channel and the injector nozzle by reducing its external diameter and increasing its length.

According to a preferred embodiment, the proximal end of the displacement element is provided with means for removably attaching the displacement element to an injector plunger. These means may be realized by means of an appropriate design of the plunger end and the displacement element. For example, the displacement element and the plunger may be made to cooperate with each other by means of a positive plug-in connection. An advantageous embodiment envisages the displacement element being designed as a piston and provided at its proximal (rear) end with a cut-out, a blind hole for example, with which the plunger can engage. But it is also conceivable for the rear side of the piston to be designed as a flat surface. In that case the piston may already be inserted when the lens carrier is loaded into the loading channel. Accordingly, plunger and piston may come into contact with their respective end faces.

The loading channel of the lens carrier is preferably cylindrical and consists of two half-shells that can be connected to each other. These can be connected to each other on one side by means of a film hinge. In order to permit better handling of the lens carrier, preferably rectangular wings are formed on the half shells. In the folded condition these wings will come to lie flat against each other. When a viscous lubricant is used, the loading channel will be sealed by the wings in the circumferential direction at least in a liquid-tight manner. Advantageously, a snap closure will be provided on the half-shells. An advantageous embodiment envisages the lens carrier and the displacement element being conserved in a sterile package. The sterile package may be opened immediately prior to the implantation to load the lens, together with the lubricant, in the lens carrier. The set is normally intended for once-only use.

The present invention also relates to an implantation set with a lens carrier or cartridge characterized by an injector with means for accommodating a lens carrier or cartridge and a mobile plunger that can be introduced into the loading channel. Preferably, the length of the plunger and the displacement element are so chosen that the displacement element will not be pushed out of the injector nozzle when the plunger is fully inserted. This set has the advantage that the plunger can be pushed fully home without having to fear that the displacement element with the lens might be pushed out.

A particular embodiment envisages a set characterized by the fact that the lens is arranged in the lens carrier in a stress-free state and the lens carrier, together with the lens and possibly also a holder for the lens carrier, are jointly conserved in a sterile package until they are used.

As mentioned at the beginning, during the operation the lens is introduced in a folded condition into the capsular bag, where it then re-assumes its original form. It is not possible for the lens to be left in a folded condition for a longish period of time prior to its use, because in that case it would not return to its original form after implantation. For this reason, the lens is conserved in the lens carrier in a stress-free condition right up to its use. The lens carrier consists of a relatively expensive biocompatible material, because during conservation together with the lens it must not give out any kind of harmful substance that could be absorbed by the lens. However, since the dimensions of the lens carrier are relatively small, the incidence of the price of the materials for the lens carrier is not very great. The primary and essential characteristic of the lens carrier material is therefore its behaviour during conservation, while price, strength and gliding properties play only a subordinate part.

When the described set is being used, the lens does not have to be handled. It is therefore not necessary for the lens to be inserted in the cartridge with any kind of tool. All that has to be done, rather, is to remove the lens carrier together with lens accommodated within it from the sterile package and insert it in the injector.

It is important that the folding of the lens should always be effected in the same direction without any special action of the operator. Advantageously, therefore, the lens carrier will be provided with means that assure the folding of the lens in a predetermined direction. As a simple means for folding the lens in a predetermined direction, it is possible to provide each half-shell with a longitudinal groove, and this in such a manner that the longitudinal groove will come to be situated close to the side of the half-shell opposite the film hinge, so that it can grip the edge of the lens. The longitudinal grooves are therefore arranged away from the centre of the half-shells. It has been found that this assures with great certainty that the lens will always be folded in the desired direction.

During the conservation of the lenses the half-shells in their unfolded condition are advantageously arranged in such a manner that the greatest internal width between the half-shells corresponds to the lens diameter of preferably 5 to 7 mm. This has the advantage that when the lens carrier with the lens is removed from the package, the lens cannot fall out of the lens carrier. The half-shells may therefore be arranged at an angle of between 60 and 150 degrees, preferably 90 degrees, with respect to each other.

The invention also relates to a set that is provided with an injector, especially a single-use injector, with an injector body and a plunger provided with a plug. It proves advantageous to provide such a set for the injection of the lens. This constitutes a guarantee that no erroneous attempt will be made to inject the lens with an unsuitable injector.

Advantageously, the single-use injector consists of an injector casing that consists of two parts and into which it is possible to insert a nozzle part provided with a channel that tapers conically in the direction of the nozzle. The nozzle part may be made of a material with a small sliding friction and have dimensions that are correct for the lens contained in the set. Such an injector can be produced at a small cost.

The injector housing will advantageously be provided with an inlet to a space situated upstream of the opening into which the lens carrier is inserted prior to the operation. The entire space and the remaining hollow in the lens carrier can be filled with a viscoelastic liquid through this inlet.

Advantageously, the lens carrier with the lens conserved inside it in a stress-free condition will be held in the package, a bottle for example, by a holder. With this holder the lens can therefore be easily removed from the package. Fixing means for the lens carrier may be formed integrally with the holder and the lens carrier may be provided with engagement means, a cog formed on a wing for example, to interact with the fixing means. The fixing means will advantageously consist of an opening in the holder of such size that the lens will be held in the lens carrier in a stress-free condition. Advantageously, at least one lateral wall of the opening will be provided with a recess with which an upstand of the lens carrier can engage. With a view to securing the lens carrier, there may be provided a safety element that can be slid between the wings of the lens carrier. A stop on the bottom of the opening of the holder and a stop on the safety element may be provided for the axial positioning of the lens in the lens carrier. Advantageously, the holder will be designed as a closure, a stopper for example, or as a part of the closure of the package.

The invention will now be described in greater detail and with reference to the figures, of which:

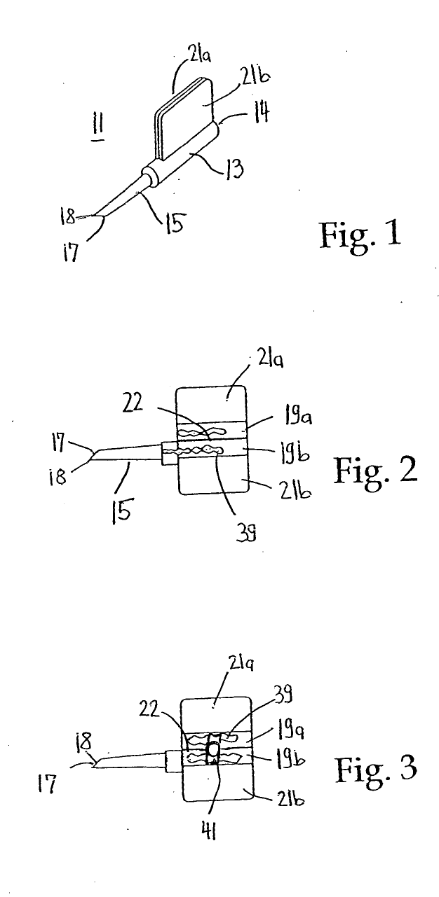

FIG. 1 shows a perspective view of a lens carrier to accommodate an intraocular lens;

FIG. 2 shows a plan view of a lens carrier partially filled with lubricant in the unfolded position;

FIG. 3 shows a plan view of the unfolded lens carrier of FIG. 2 with a lens inserted in the loading channel;

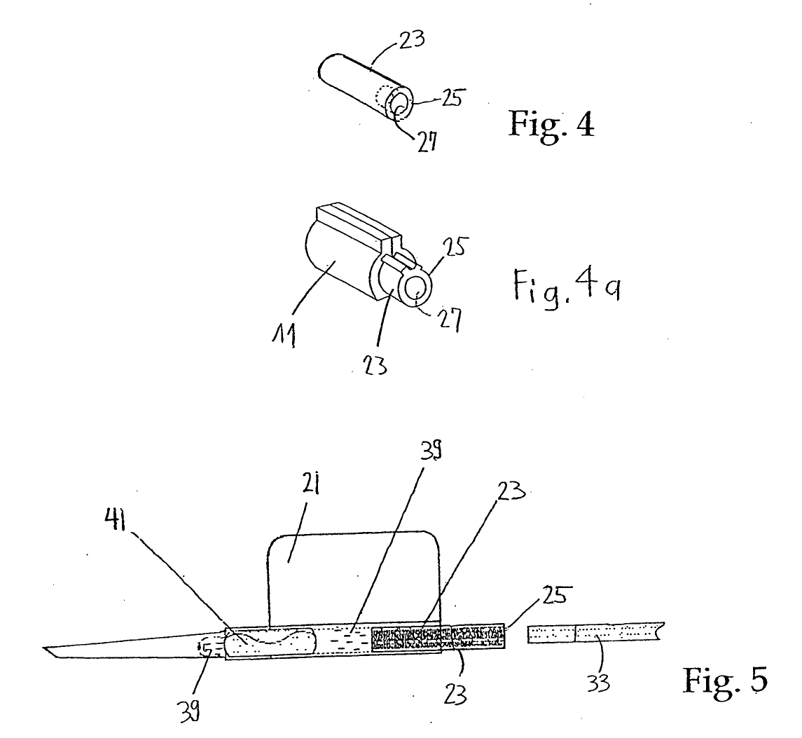

FIG. 4 shows a perspective view of a displacement element designed as a piston;

FIG. 4a shows a displacement element as in FIG. 4 for use with a lens carrier in accordance with FIGS. 11, 12 or 13;

FIG. 5 shows a schematic longitudinal section through a cartridge loaded with an intraocular lens and lubricant;

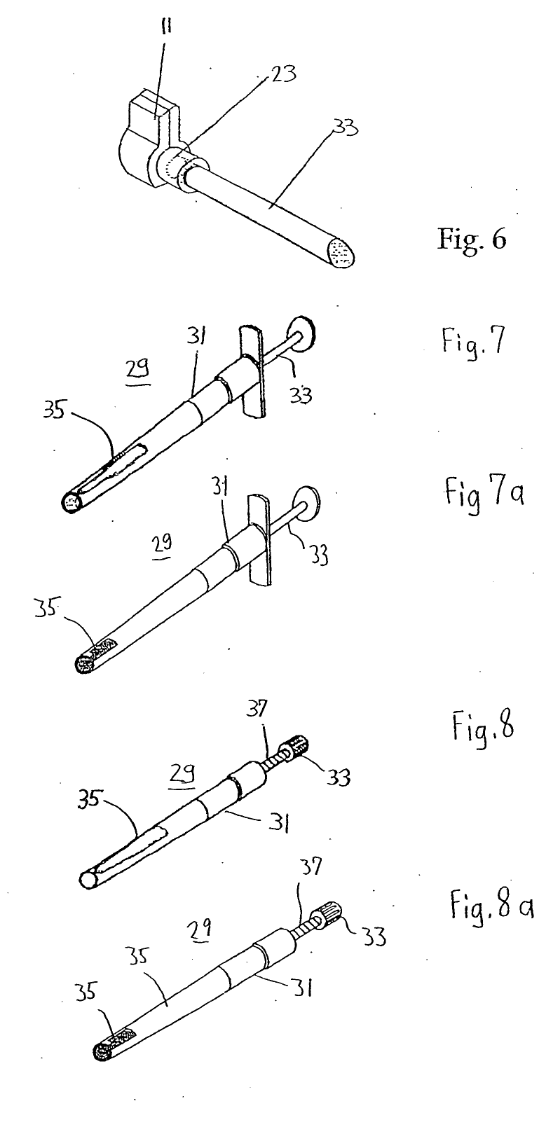

FIG. 6 shows a partial perspective view of the rear part of the loaded lens carrier and the plunger inserted in it;

FIG. 7 shows an injector;

FIG. 7a shows an injector as in FIG. 7, but for introducing the lens carrier from the front end;

FIG. 8 shows a second embodiment of an injector with a plunger with a screw thread;

FIG. 8a shows an injector as in FIG. 8, but for introducing the lens carrier from the front end;

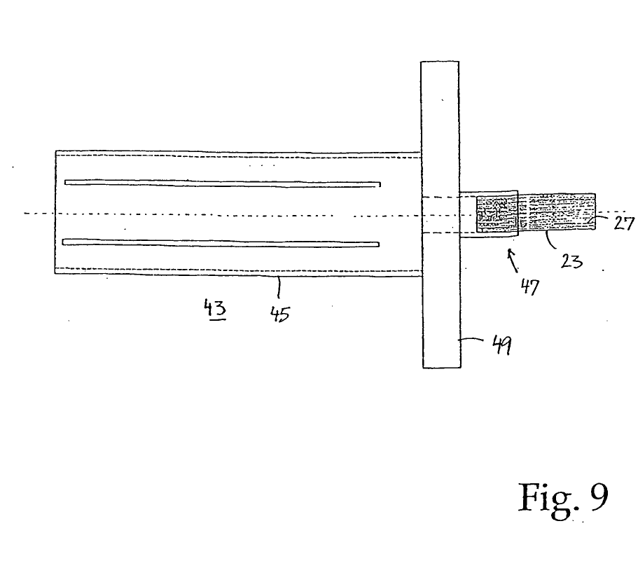

FIG. 9 shows a side elevation of a holder for fitting the piston onto a plunger, complete with a piston;

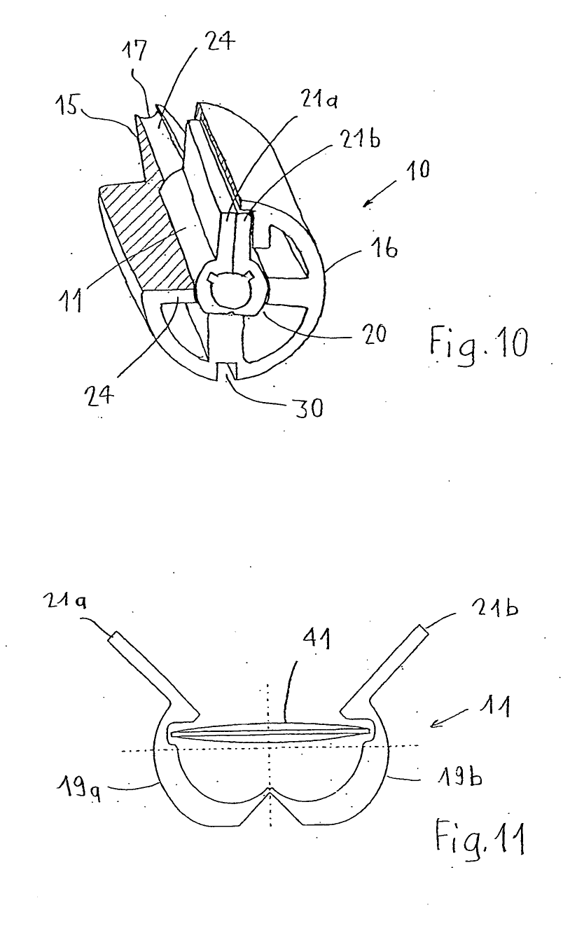

FIG. 10 shows a perspective view of a cartridge consisting of a cartridge part and an inserted lens carrier;

FIG. 11 shows an axial view of the unfolded lens carrier with a lens arranged inside it;

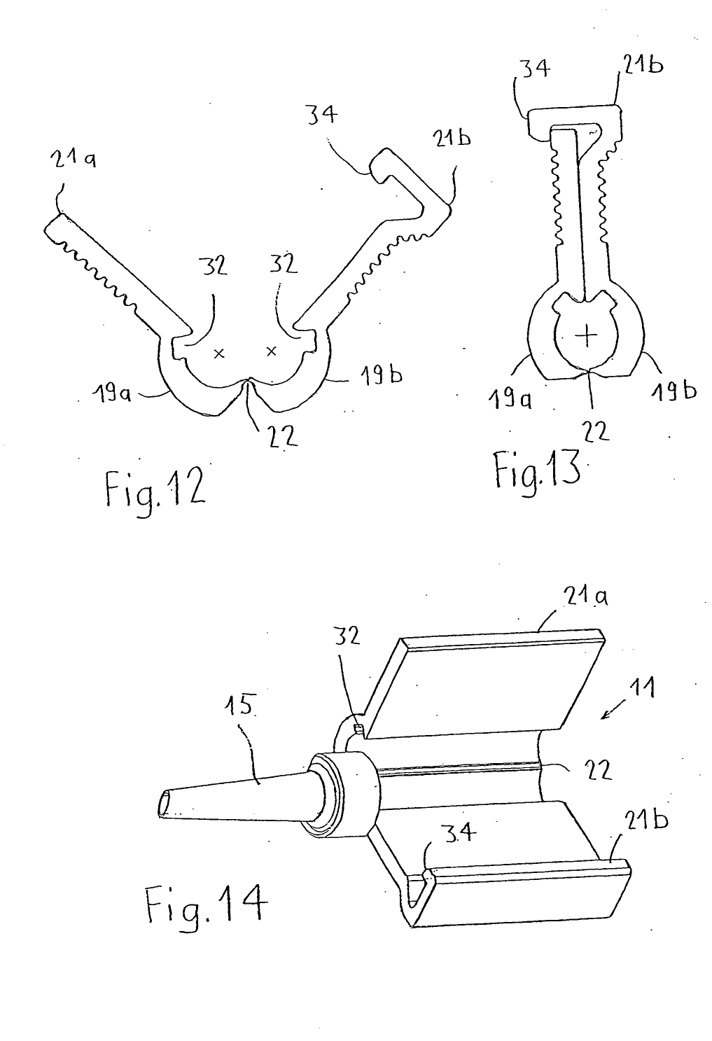

FIG. 12 shows an axial view of a preferred embodiment of an unfolded lens carrier;

FIG. 13 shows an axial view of the folded and locked lens carrier of FIG. 12;

FIG. 14 shows an embodiment of a lens carrier with an injector nozzle arranged on it (attached thereto);

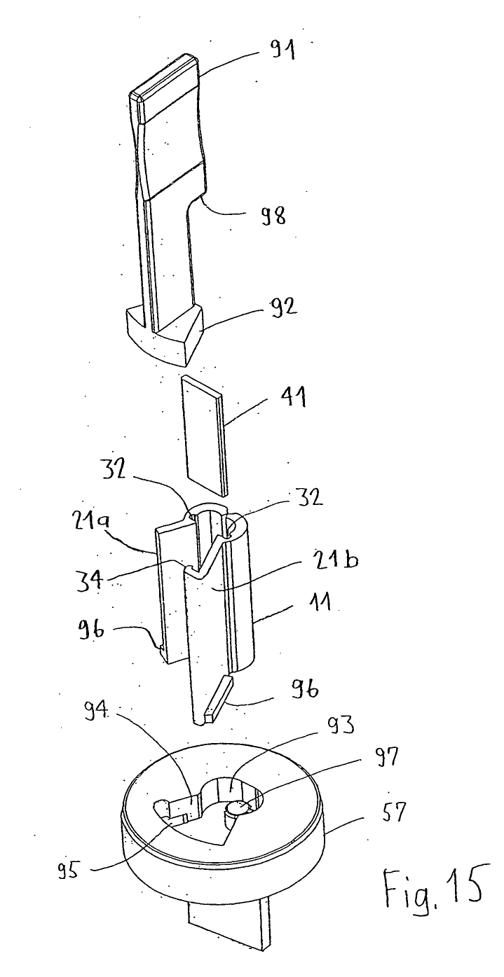

FIG. 15 shows a preferred embodiment of the holder;

FIG. 16 shows a preferred embodiment of the injector after the insertion of the lens carrier and the filling of the injector with the viscoelastic solution;

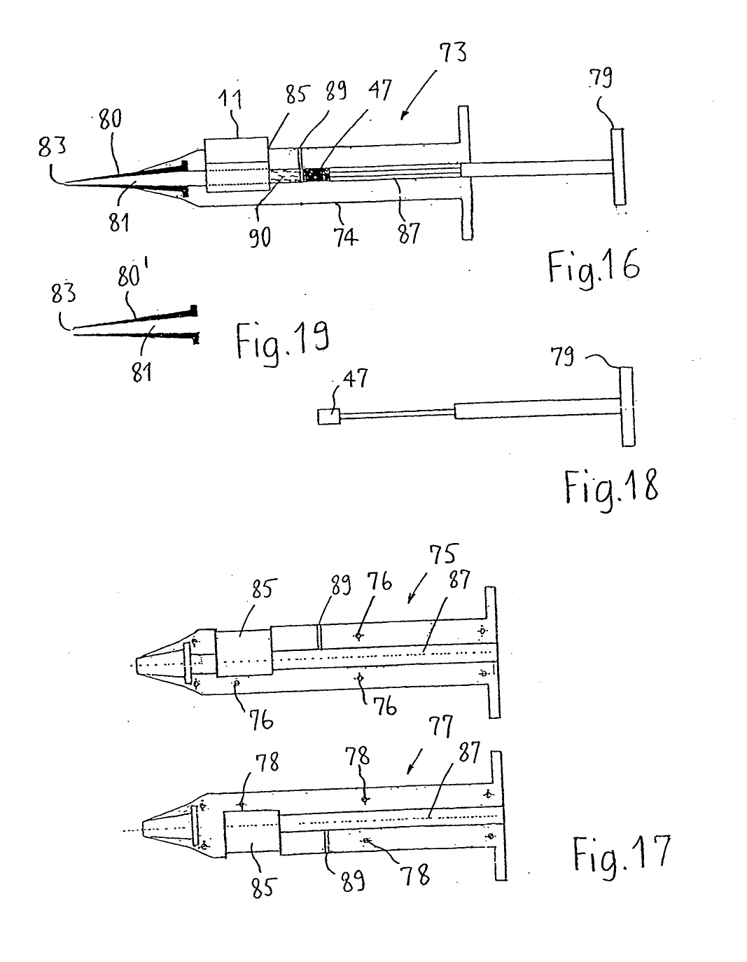

FIG. 17 shows the two halves of the housing of the injector of FIG. 16,

FIG. 18 shows the plunger with the stopper placed on it;

FIG. 19 shows a nozzle part with a larger diameter than in FIG. 16.

The lens carrier shown in FIGS. 1 to 3 consists of cylindrical loading channel 13 with a proximal entry opening 14 and an injector nozzle adjacent to the end of the loading channel 13 and aligned with it. The injector nozzle 15 tapers conically in the direction of its tip. It has a chamfered tip 18 with a distal exit opening 17. The chamfered tip 18 makes it possible for the injector nozzle to be introduced into the eye even through a small incision having a width of 2 to 3 mm.

According to the shown embodiment, the lens carrier 11 can be pivotally unfolded and folded. The loading channel 13 consists of two cylindrical half-shells 19a, 19b that are joined together on one side by means of a film hinge 22. To permit ready handling of the lens carrier, the half-shells 19a, 19b are provided with respective rectangular wings 21a, 21b integrally attached to them.

Preferred embodiments of lens carriers 11 will be described below with reference to FIGS. 12 to 13.

According to the invention, there is provided a displacement element designed as a piston 23 (FIGS. 4 and 4a). The piston 23 is such as to correspond substantially to the diameter and preferably also to the interior contour of the loading channel 13. The piston 23 substantially seals the loading channel 13 towards the rear (proximal end). At its rearward face 25 it is provided with a blind hole. 27 with which the plunger 33 of a known injector can engage (FIGS. 4 and 5).

An injector 29 (FIG. 7) frequently used for implantations has an elongated casing 31 in which there is enclosed an axially movable plunger. The present invention makes use of a plunger 33 that has a smaller diameter than the internal diameter of the loading channel 13. In the forward third of the casing 31 there is provided a slot-like cut-out 35 into which a lens carrier 11 can be inserted. When the lens carrier 11 is inserted in the injector 29, the loading channel 13 is aligned with the plunger 33. It is also possible to use an injector (FIG. 8) in which the plunger 33 is provided with a screw thread 37 that interacts with a corresponding internal thread of the casing 31.

As is shown by FIGS. 7a and 7b, the injectors may also be designed in such a manner that the lens carrier 11 can be inserted from in front.

The lens carrier is used as follows: The lens carrier to be loaded is removed from its sterile package and unfolded. The loading channel 13 is then filled at least partially, but preferably completely, with a viscous lubricant 39 (FIG. 2). Thereafter an intraocular lens (IOL) 41 is laid into the loading channel filled with the lubricant 39 in such a manner that the lubricant 39 will be situated both in front of and behind the lens (FIG. 3). “In front” here means closer to the distal opening 17 and ‘behind’ means nearer to the proximal entry opening 14. The lens carrier 11 is then folded. When this is done, the lens 41 becomes folded. A yielding displacement element, a piston 23 for example, is then attached to the plunger 33 of the injector 29 and the lens carrier 11 is loaded into the slot-like cut-out 35 of the injector 29. For the implantation the plunger 33 is pushed forward into the casing 31. When this is done, the piston 23 attached to the plunger 23 penetrates into the loading channel 13. Since the piston 23 substantially seals the loading channel 13 on one side, a further forward movement of the piston 23 will first displace the lubricant 39, together with the lens 41 enveloped by it, into the injector nozzle 15 and then eject it therefrom. The compressible and deformable piston 23 has the property that it will substantially adapt to the diameter of the channel. Given the compressibility and deformability of the employed material, the piston may be pushed, preferably at least partially, into the injector nozzle that tapers in the forward direction and therefore has a smaller diameter at its front end than the loading channel. When this is done, the piston, given the diminishing diameter of the injector nozzle, will increase its length correspondingly—as a general rule by a factor of at least 1.5—while maintaining its volume. For example, the loading channel may have a diameter of between 2.5 and 3 mm and the injector nozzle a diameter of less than 2 mm.

A holder 43 (FIG. 9) is provided for attaching the piston 23 to the plunger 33. The holder 43 has a grip 45 for being gripped with the fingers and a mounting 47 to receive the piston 23 made of an elastomeric material, silicone for example, having a hardness between 10 and 80 Shore, preferably between 20 and 65 Shore, and even more preferably between 25 and 40 Shore. A shield 49 is arranged between the grip 45 and the mounting 47. The mounting 47 consists of a blind hole or a through hole. The side walls of the mounting 47 are flat in cross section, so that a piston 23 arranged within the mounting will touch the side walls of the mounting 47 in three or four regions of limited area. The holder 43 is designed as a hollow cylinder. The grip 45 is provided with ribs to permit the user to grip it more readily. The shield 49 is configured in such a manner as to make it bear against a flat subgrade at right angles to the axis of the hollow cylinder of the grip 45. This makes it impossible for the holder to roll away on an inclined plane.

When the piston 23 is to be attached to the plunger 33, the holder 43 with the piston 23 inserted in the mounting 47 is taken into one hand and the plunger 33 into the other. The blind hole 27 on the piston 23 is arranged at the end of the piston 23 that faces away from the grip 45. The forward end of the plunger is then introduced into the blind hole 27 on the piston 23. The plunger with the attached piston 23 can now be separated from the holder 43. The plunger 33 with the attached piston 23 can now be introduced into the loading channel 13.

FIG. 10 shows a cartridge 10 for-use with an injector. The set for the cartridge 10 contains a lens carrier 11 and a lens 41 arranged therein in a stress-free condition as in FIG. 11, as well as the cartridge part 16. Advantageously, however, the lens carrier is designed as will later be described with reference to FIGS. 13 and 14. The lens carrier consists of a biocompatible material capable of being autoclaved. The lens holder 11 and the lens 41 are jointly conserved in a sterile package up to the moment when they are used. If the lens is made of hydrophilic material, the joint conservation will be effected in a liquid. The lens carrier 11 can be folded together with the lens 41. The lens carrier 11 consists of two half-shells 19a, 19b that are joined to each other by means of a film hinge 22. The shape of the lens carrier 11 is such as to make it assume a preferably hollow cylindrical form after it is has been folded (FIG. 1). The half-shells 19a, 19b are arranged at an angle of about 90 degrees. But this angle can vary in the range between about 60 and 90 degrees. Each half-shell is provided with a wing 21a, 21b that facilitates the folding of the lens carrier 11.

The cartridge part 16 (FIG. 10) consists of a material with good sliding properties for the lens, has a cylindrical form and is provided with an injector nozzle 15. A reception opening 20 extends coaxially with the channel 24 of the injector nozzle 15. This channel 24 has a slight conical taper in the direction of the nozzle 17. In the shown embodiment of the cartridge part 16 the reception opening 20 has a larger diameter than the outer diameter of the lens carrier after it has been folded. Ribs 24′, 26, 28 extend inwards to receive the lens carrier 11, a groove being provided for accommodating the wings 21a, 21b of the lens carrier 11.

The set may also contain the displacement element 23 (FIG. 4) made of an elastomeric material, silicone for example.

When the set is produced, the lens 41 is laid in the lens carrier 11. The latter is to all intents and purposes in a stress-free state. The lens carrier 11 with the lens 41 is then enclosed in a sterile package. As a general rule, the sterile package consists of polycarbonate or glass and, in the case of hydrophilic lenses, is filled with a conservation liquid. In this sterile package the lens 41 together with the lens carrier 11 can be sterilized in an autoclave at 140°.

On the occasion of the operation the lens carrier 11 with the lens 41 is taken out of the sterile package. For folding purposes, a suitable tool can be used to carefully grip the lens carrier by the wings 21a, 21b and fold it.

The folded lens carrier 11 is then inserted in the reception opening 20 of the cartridge part 16. The cartridge 10 is then ready for insertion in the injector. A groove 30, a rib or some other suitable provision assures the correct angular position of the cartridge 10 and therefore also of the lens 41 in the injector.

It is desirable that the folding of the lens carrier should always fold the lens in a predetermined direction. As is illustrated by FIGS. 12 and 13, a [particular] embodiment of the lens carrier 11 envisages each half-shell 19a, 19b being provided in the vicinity of the wing with a longitudinal groove 32 to accommodate the edge of the lens 41. The arrangement of the longitudinal grooves 32 away from the centre of the half-shells assures that the lens 41 will always fold in the direction of the hinged joint 22. A snap closure device 34 assures that the lens carrier 11 will remain in the folded position after the lens 41 has been folded.

The lens carrier 11 in accordance with FIGS. 12 and 13 does not have an injector nozzle and for use purposes is provided with a separate injector nozzle (80, 80′), as can be seen from FIGS. 16 and 19. FIG. 14, on the other hand, shows a lens carrier 11 similar to the one of FIGS. 12 and 13, but also provided with an injector nozzle 15 like the lens carrier of FIGS. 1 to 3.

FIG. 15 illustrates a holder device by means of which the lens carrier 11 and the lens 41 conserved in it in a stress-free condition are kept firmly in position within the package, a bottle for example.

This holder device consists essentially of the holder 57 and the safety element 91. The safety element is made of a biocompatible plastic material. The holder 57 may be designed as a stopper for closing a package, a bottle for example. The stopper may be made of silicone or some other flexible biocompatible material. But it is also possible for the holder 57 to be designed as part of a closure, a screwed closure for example. It may be made of a biocompatible plastic material.

The opening 93 in the holder 57 serves as the fixing means for the lens carrier 11. At least one side wall 94 of the opening 93 is provided with a recess 95 with which the upstand 96 of the lens carrier 11 can engage. In all other respects the lens carrier is designed as has already been described with reference to FIGS. 12 and 13.

The opening 93 is so dimensioned that the lens 41 will be held in the lens carrier 11 in a stress-free condition. The opening 93 is therefore appropriately dimensioned to take account of the size of the lens 41. Nevertheless, the same lens carrier 11 may be used for various lens sizes.

When a lens 41 is to be inserted in the holder 57 and/or the lens carrier 11, the lens carrier 11 is first inserted in the holder 57 and the lens 41 is then slid into the longitudinal grooves 32 with help of forceps, after which the safety element 91 is placed between the wings 21a, 21b of the lens carrier. The head 92 of the safety element 91 is dimensioned in accordance with the opening 93 and therefore in accordance with the resulting angle of aperture between the wings 21a, 21b of the lens carrier 11. After the safety element 91 has been slid into position, rest friction prevents the safety element 91 from falling out. The safety element 91 also assures that the lens 41 will be axially positioned between the stop 97 on the bottom of the opening 93 and the stop 98 of the safety element. The holder 57 can now be turned through 180 degrees and inserted in a container.

When the lens 41 is to be taken out of the holder 57 during the operation, the lens carrier 11 has to be gripped by the wings 21a, 21b, pressing them together. The lens 41 will thus be folded and, following closure of the snap device, will remain safely in this position. Even if the lens holder 11 should slip out of the hand of the operator when it is removed from the holder 57 or inserted in the injector, the lens will remain protected in its folded condition. Therefore, always provided that the slipped lens carrier has remained in a sterile region, the operation can be continued in a problem-free manner and without adverse consequences.

FIG. 16 illustrates a preferred embodiment 73 of an injector. The latter may be designed as a single-use injector. The housing 74, made of plastic material for example, consists of the housing halves 75, 77 (FIG. 17). One of the housing halves, 75 for example, has plugs 76 that can engage with recesses 78 in the other housing half 77. The injector 73 comprises a plunger 33 with a displacement element made of the previously described elastomeric material. A nozzle part 80 is inserted in the housing 74. The nozzle part contains a channel 81 that tapers conically in the direction of the nozzle 83. The diameter of the nozzle 83 will be larger or smaller in accordance with the type and size of the lens that is to be injected. FIG. 19 thus shows a nozzle part 80′ in which the diameter of the nozzle 83 is greater than in FIG. 16. The injector housing 74 is provided with a lateral opening into which the lens carrier 11 can be inserted. It is also provided with an inlet channel 89, arranged at right angles to the longitudinal channel, for the viscoelastic liquid that fills the space 90 and the hollow in the lens carrier 11.

It is also possible for the injector 73 to be designed in a manner similar to FIG. 7a to permit insertion of the lens carrier 11 (FIG. 14) from in front.

During the operation the lens carrier 11 with the lens 41 is removed from the sterile package, after which it is folded together with the lens 41 and inserted in the injector 73. The viscoelastic liquid or gel is then introduced through the channel 89. At this point the injector 73 is ready for injecting the lens 41 into the eye to be treated. When the plunger 79 is pushed home, the lens 17 is injected through the channel 81 into the previously emptied capsular bag. As already mentioned in the introduction, the lens 41 is transported through the channel of the nozzle part 80 without any direct contact, or at least greatly reduced contact, because the viscoelastic liquid serves not only as a lubricant, but also as a hydraulic means.

Summarizing, one may note the following:

With a view to inserting an intraocular lens 41 in the capsular bag of the eye, an overpressure is produced in order to push a lens 41 floating in a lubricant 39 out of the injector nozzle. The compressible and deformable piston 23 can adapt to the nozzle channel tapering in the forward direction. The lens 41 is further folded on its way and has a very small diameter when it reaches the end of its path. The incision can therefore be kept very small. The set contains a lens carrier 11 and a lens 41. The lens 41 is kept in the lens carrier 11 in a stress-free condition. The lens 41 and the lens carrier 11 are preferably retained in a holder 57 and, until actual use, conserved in a sterile package and, more precisely, in the case of a hydrophilic lens, in a liquid that prevents the lens from drying out. During the operation the lens carrier 11, together with the lens 41 conserved in it, is removed from the package, folded and inserted in the injector. Thereafter a viscoelastic liquid is introduced through the channel 89. The lens can now be injected into the capsular bag of the eye that is to be treated. When this is done, there is no danger of the lens being damaged during folding and injection.

1. A method of preparing an intraocular lens and inserting it by means of an injector nozzle into the capsular bag of the eye to be treated, wherein the lens is deposited in a lens carrier, which is provided with a loading channel and a proximal opening after which the lens carrier with the lens is folded and loaded into an injector and the lens is pushed out through the injector nozzle, characterized in that, for the purpose of ejecting the lens, an overpressure is produced on the side of the proximal opening and the lens, together with a least a part of the lubricants, is displaced from the loading channel into the injector nozzle and eventually ejected therefrom.

2. A method in accordance with claim 1, characterized in that the cartridge with the lens 44 is loaded into an injector, provided with a mobile plungers, that a displacement element serving as piston is introduced or inserted in the proximal opening, and that the lens, together with a part of the lubricants, is displaced from the loading channel into the injector nozzle by means of the overpressure exerted by the movement of the displacement element and ejected from the nozzle.

3. A method in accordance with claim 1,
characterized in that the quantity of lubricant employed is such as to have the lens enveloped in a plug of lubricant.

4. A method in accordance with claim 1, characterized in that the displacement element is placed on the plunger and then introduced through the opening into the loading channel.

5. A method in accordance with claim 1, characterized in that the lens is laid into the lens carrier and is then sterilized and packed together with the latter.

6. A set for receiving and inserting an intraocular lens, with a lens carrier with a loading channel provided with an opening for a lens and an injection nozzle axially attached to the loading channel, or capable of being so attached, as well as means for the production of an overpressure that can be attached to the opening of the loading channel or inserted therein.

7. A set in accordance with claim 6, characterized in that as means for the production of an overpressure there is provided a displacement element displacement element that can be inserted in the loading channel.

8. A set in accordance with claim 7, characterized in that the displacement element is a piston made of compressible plastic material, preferably a silicone piston.

9. A set in accordance with claim 7, characterized in that at the proximal end of the displacement element there are provided means for removably attaching the displacement element to a plunger of an injector.

10. A set in accordance with claim 9, characterized in that the attachment means is a recess or a blind hole.

11. A set in accordance with claim 8, characterized in that the displacement element is made of an elastic and/or compressible material, preferably a plastic material, for example silicone or foamed plastic material.

12. A set in accordance with claim 6, characterized in that the lens carrier can be folded together with the lens.

13. A set in accordance with claim 6, characterized in that after folding the lens carrier preferably has the form of a hollow cylinder.

14. A set in accordance with claim 6, characterized in that the loading channel tapers conically in the direction in the direction of the outlet opening.

15. A set in accordance with claim 6, characterized in that the loading channel of the lens carrier is cylindrical and consists of two half-shells that are or can be connected to each other by means of a hinge, a film hinge for example.

16. A set in accordance with claim 6, characterized in that wings are integrally attached to the half-shells.

17. A set in accordance with claim 8, characterized in that lens is arranged in a stress-free condition in the lens carrier and that the lens carrier together with the lens and possibly also a holder for the lens carrier are conserved in a sterile package until they are used.

18. A set in accordance with claim 17, characterized in that the lens carrier is provided with means that assure the folding of the lens in a predetermined direction.

19. A set in accordance with claim 18, characterized in that, as means for folding the lens in a predetermined direction, the interior wall of each of the half-shells, close to the end opposite the hinge, is provided with a longitudinal groove that can grip the edge of the lens.

20. A set in accordance with claim 16, characterized in that a snap closure is formed on the half-shells.

21. A set in accordance with claim 6, characterized in that it contains an injector, especially a single-use injector, with an injector housing and a plunger provided with a displacement element and that the injector housing is provided with a longitudinal channel for accommodating the plunger and an opening into which the lens carrier with the lens can be inserted.

22. A set in accordance with claim 21, characterized in that the injector has an injector housing consisting of two parts into which there is inserted a nozzle part with a channel that tapers conically in the direction of the nozzle.

23. A set in accordance with claim 21, characterized in that the injector housing is provided with an inlet into a space, said inlet being situated upstream of the opening for the lens carrier.

24. A set in accordance with claim 17, characterized in that the lens carrier with the lens conserved in it in a stress-free condition is retained in the package by means of a holder provided with a fixing means.

25. A set in accordance with claim 24, characterized in that the holder is provided with fixing means for the lens carrier and the lens carrier is provided with locking means, an upstand arranged on one of the wings for example, to interact with the fixing means.

26. A set in accordance with claim 24, characterized in that an opening in the holder serves as fixing means, said opening being so dimensioned that the lens is held in the lens carrier in a stress-free condition.

27. A set in accordance with claims 26, characterized in that at least one side wall of the opening is provided with a recess with which an upstand of the lens carrier can engage.

28. A set in accordance with claim 25, characterized in that there is provided a safety element that can be slid between the wings of the lens carrier in order to secure the lens carrier engaged with the holder.

29. A set in accordance with claim 25, characterized in that a stop is provided on the bottom of the opening of the holder and on the safety element to assure the axial positioning of the lens in the lens carrier.

30. A set in accordance with claim 24, characterized in that the holder constitutes the closure, a stopper for example, or a part of the closure of the package.

31. A set in accordance with claim 6, characterized in that the channel of the injector nozzle tapers in the direction of the outlet opening.

32. A set in accordance with claim 21, characterized in that the length of the plunger and the displacement element is chosen in such a manner that the displacement element will not be pushed out of the injector nozzle when the plunger is moved into its most forward position.

33. A set in accordance with claim 7, characterized by a holder for holding the displacement element and for ounting the displacement element on the plunger.

34. A set in accordance with claim 7, characterized in that the displacement element and/or the material from which the displacement element is made has a hardness of between 10 and 80 shore, preferably between 20 and 65 Shore, and even more preferably between 25 and 40 Shore.

35. A method of injecting an intraocular lens into the eye to be treated, characterized in that the lens carrier is removed from the sterile package, folded and inserted in the injectors, that the residual hollow in the lens carrier is filled with a gel, and that the lens is injected through the channel in the nozzle part into the previously emptied capsular bag of the eye to be treated when the plunger provided with an elastomeric displacement element is pushed home.
